(19) United States
(12) Patent Application Publication
(10) Pub. No.: US 2008/0269325 A1
(43) Pub. Date: Oct. 30, 2008

(54) Methods to Relieve Pain

(75) Inventors: Andrew Sven Cracroft Rice, London (GB); Severine Vandvoorde, Brussels (BE); Didier Michel Lambert, Brussels (BE)

Correspondence Address: WILMERHALE/BOSTON, 60 STATE STREET, BOSTON, MA, 02109, US

(73) Assignee: Imperial Innovations Limited, London, EN (GB)

(21) Appl. No.: 10/590,131

(22) PCT Filed: Feb. 18, 2005
(86) PCT No.: PCT/GB05/00597

§ 371 Date: Nov. 14, 2007

(30) Foreign Application Priority Data
Feb. 19, 2004 (GB) 0403629.9

(51) Int. Cl. A61K 31/26; A61P 29/00

(52) U.S. Cl. 514/515

(57) ABSTRACT

The invention is directed to a method of providing pain relief to a patient by administering to the patient an effective amount of a compound of Formula (I), which is represented by as RC(O)—NH—(CH₂)ₙ—CH═CH₂, wherein R represents C₁₋₂₀ alkyl, C₂₋₂₀ alkenyl or C₂₋₂₀ alkynyl; and n is an integer from 0 to 3.

The present invention relates to pain relief and, in particular, compounds for use as analgesics.

The control and relief of pain is an important branch of medicine. Pain may come about both as a result of disease or injury as well as a result of medical treatment such as chemotherapy. In each case, it is important to alleviate the pain as much as possible so as to enable the sufferer to function normally.

Pain is a complex sensation and is the most common symptom of disease. Pain may be nociceptive pain, which is due to a noxious stimulus (chemical, thermal, mechanical etc.) that activates pain receptors, or neuropathic pain, which results from disease of the central or peripheral nervous system. Pain may also be classified as being acute or chronic in nature. Acute pain is usually a result of injury (e.g. trauma or disease), it lasts a short time and is typically resolves as the injured tissue heals or soon after. Chronic pain is usually defined broadly and arbitrarily as a pain persisting for over one month beyond the resolution of an acute tissue injury, pain persisting or recurring for more than three months, or pain associated with tissue injury that is continued or progressed (The Merck Manual, 1999).

Several pain syndromes are difficult to classify according to these criteria. These include, for example, chronic headache and continuous acute pain produced by the invasion of body tissues in malignant diseases.

Two neural pathways relating to pain act concurrently in the body: (1) a sensory pathway which senses tissue damage and subsequently produces a feeling of pain; (2) an analgesic pathway which reduces the feeling of pain and prevents the flow of information about the pain to the central nervous system (CNS), thus allowing the organism to maintain it's normal activity in spite of an injury. Since these are different pathways, they are affected by different substances. For example, aspirin and lidocaine are active on the peripheral sensory pathway, while morphine and related substances are active on the analgesic system.

Opioids, such as morphine and related opioid compounds are considered as the most potent analgesic agents and are often required for relief of severe pain. However, these narcotic drugs have the severe drawback of leading to dependence and addiction. In addition, patients treated with opioids tend to develop tolerance to the drug, which leads to increasing dosage of the drug being needed for exerting the analgesic effect and to subsequent withdrawal symptoms. Further side effects associated with opioid drugs include nausea, sedation and respiratory depression.

Nonopioid analgesics, e.g. cyclooxygenase inhibitors such as acetaminophen (paracetamol) and other nonsteroidal anti-inflammatory drugs (NSAIDs) are often effective for treatment of mild to moderate pain. Anti-inflammatory agents of the NSAID class such as acetylsalicylic acid (aspirin), indomethacin, diclofenac and benzydamine have been used as analgesics in pain associated with trauma and inflammation. Common side effects of the NSAID class of drugs include gastrointestinal irritation and ulceration, blockade of platelet aggregation, renal dysfunction and hepatic damage.

Another major class of analgesics is the local anaesthetics that block sodium channels. Compounds of this class, e.g. lidocaine, when topically applied to the spine, have been found effective for control of pain after surgery or trauma, but require expertise and infrastructure to administer and monitor properly. Systemic infusion of lidocaine can reduce acute pain, but requires continuous monitoring so that resuscitation from seizures or apnea can be performed immediately.

Additional classes of analgesics include local anaesthetics, NMDA receptor antagonists, antidepressants and anticonvulsants.

In addition to the above classes of analgesics, strong laboratory evidence now underwrites anecdotal claims of cannabinoid analgesia in inflammatory and neuropathic pain (Rice et al (2003) Cannabinoids and pain. In: Dostrovsky, Carr and Kolzenburg Eds. Seattle: IASP Press, 437-468). Recent advances have dramatically increased our understanding of cannabinoid pharmacology: the psychoactive constituents of Cannabis sativa have been isolated, synthetic cannabinoids described and an endocannabinoid system identified, together with its component receptors, ligands and their biochemistry. Sites of analgesic action have been identified in brain, spinal cord and the periphery, with the latter two presenting attractive targets for divorcing the analgesic and psychotrophic effects of cannabinoids.

To date two cannabinoid receptors have been cloned, CB₁ in 1990 (Matsuda et al (1990) Nature 346, 561-564) and CB₂ in 1993 (Munro et al (1993) Nature 365 61-65). Both are G-protein coupled receptors and possess the typical structure consisting of an extracellular N-terminal domain, seven transmembrane helices and an intracellular C-terminal domain.

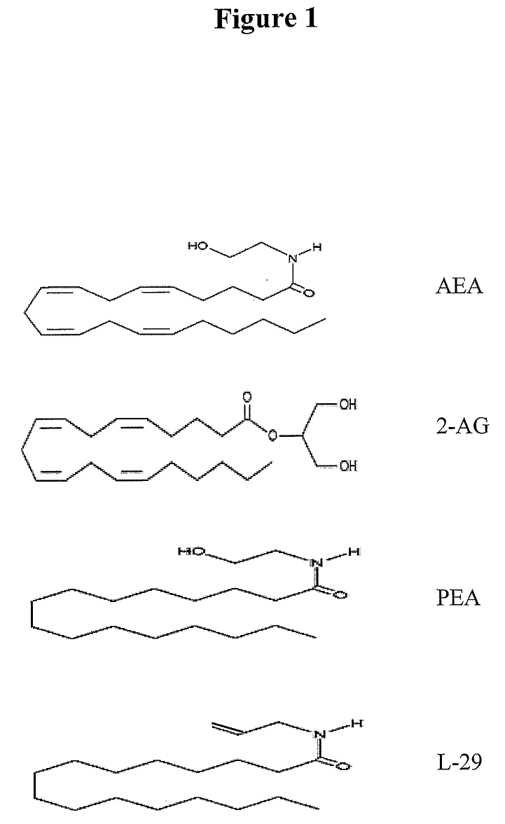

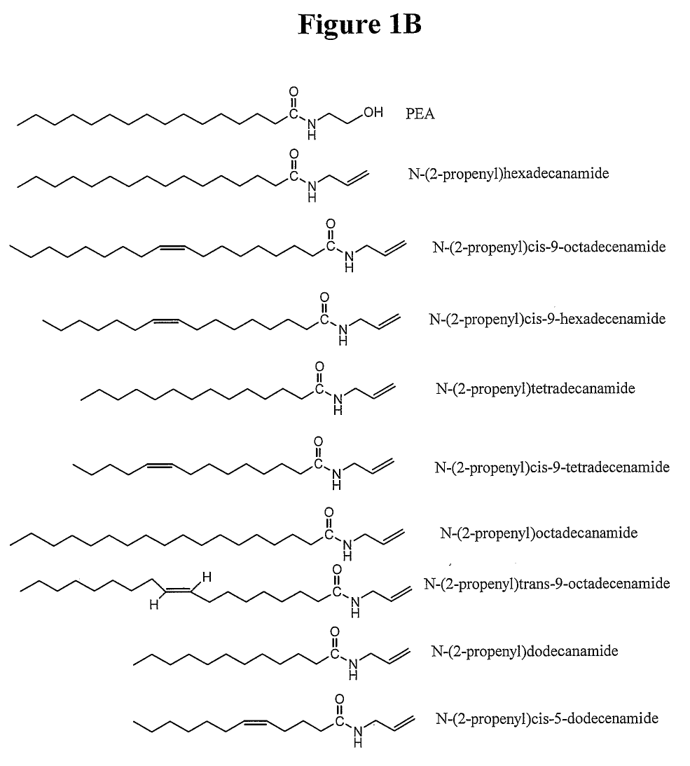

Several endogenous ligands that bind to CB₁ receptors have been discovered: the two prototypical ligands are anandamide (arachidonylethanolamide, AEA) and 2-arachidonylglycerol (2-AG), both derivatives of a long chain fatty acid (FIG. 1). AEA binds to the CB₁ receptor and evokes the classical tetrad of cannabinoid effects, ie analgesia, hypomotility, catalepsy and hypothermia, as well as having effects on memory processes, spasticity and cell proliferation. It also binds to the CB₂ receptor, where it does not evoke biologically significant activity, and is a weak agonist at the vanilloid receptor (VR1). 2-AG also has cannabimimetic effects and is found endogenously at much higher concentrations than AEA. It binds only weakly to CB₁, but is a full agonist, and also binds to CB₂ receptors. It has been suggested that 2-AG is the endogenous ligand at the CB₂ receptor (Hillard (2000) Prostaglandins Other Lipid Mediat 61, 3-18).

Palmitoylethanolamide (PEA) (FIG. 1) is another endogenous cannabimimetic compound that is a fatty acid derivative. It possesses only limited binding at CB₁ or CB₂ receptors, yet some of its effects are prevented by the CB₂ receptor antagonist SR144528 (SR2) (Calignano et al (1998) Nature 394 277-281; Farquhar-Smith et al (2002) Pain 97, 11-21; Farquhar-Smith and Rice (2001) Anesthesiology 94(3), 507-513; Farquhar-Smith and Rice (2003) Anesthesiology 99, 1391-1401).

PEA has anti-inflammatory and analgesic effects in vivo (Calignano et al (1998) Nature 394 277-281; Jaggar et al (1998) Pain 76 189-199; Lambert et al (2001) Epilepsia 42 321-327; Farquhar-Smith et al (2002) Pain 97, 11-21; Farquhar-Smith and Rice (2001) Anesthesiology 94(3), 507-513; Farquhar-Smith and Rice (2003) Anesthesiology 99, 1391-1401), and there are several theories on how this effect is mediated: PEA may be enhancing the effects of other endogenous ligands that bind to the CB₂ receptor (the entourage effect); or it may be binding to a new non-CB₁/CB₂ receptor at which SR2 also acts as an antagonist.

Fatty acid amide hydrolase (FAAH) is an enzyme that degrades many fatty acid amides including the endocannabinoids (Boger et al (2000) Bioorganic and Medicinal Chemistry Letters 10 2613-2616). It is distributed widely in the brain (Egertova et al (1998) Proc R Soc Lond 265 2081-2085; Tsou et al (1998) Neuroscience Letters 254 137-140; Romero et al (2002) Molecular Brain Res 100 85-93) and periphery and is known to degrade both AEA and PEA (Tiger et al (2000) Biochemical Pharmacology 59 647-653). Recent evidence has shown that FAAH has complementary localisation with CB₁ receptors in many brain regions (Egertova et al (1998) Proc R Soc Lond 265 2081-2085) including those involved in pain pathways.

As FAAH degrades fatty acid amides, including the endocannabinoids AEA and PEA, inhibiting FAAH function in vivo may have the effect that the endocannabinoids persist for longer and thus may have more prolonged effects, for example analgesic effects. Towards these goals various FAAH inhibitors have been discovered (Martin et al (2000) J Pharmacol Exp Ther 294 1209-1218; Boger et al (2000) Proc Natl Acad Sci USA 97 5044-5049) and, indeed, PEA itself has some ability to inhibit FAAH (Jonsson et al (2001) Br J Pharmacol 133 1263-1275).

Recently, a number of studies have been made of the structure-activity of PEA. Vandevoorde et al (2003) J Med Chem 46 1440-1448 synthesised analogues and homologues of PEA in which the ethanolamine head group was changed and found that none of the compounds were dramatically more potent than PEA at reducing AEA metabolism by inhibiting FAAH function.

Similarly Jonsson et al (2001) Br J Pharmacol 13, 1263-1275 synthesised a number of analogues and homologues of PEA in which both the ethanolamine head group and the fatty acid tail were modified. The authors found that there was little difference in the ability of the analogues to reduce AEA metabolism by FAAH when compared to PEA. However, two of the compounds did increase AEA uptake into the cell when compared to PEA and so may be useful as ‘entourage’ compounds.

In a further study Lambert et al (1999) Biochem Biophys Acta 1440 266-274 examined the capacity of PEA and analogues and homologues having changes in both the ethanolamine head group and the fatty acid tail to act as ligands for CB₁ and CB₂. The authors found that PEA was a weak ligand for the CB₁ and CB₂ while the analogues and homologues essentially did not bind the receptors.

WO 00/32200 considers more anandamine analogues but does not provide any indication of activity in vivo.

We have found that compounds with similarities to PEA in which the ethanolamine group has been replaced by an allylamine group can act as an analgesic in animal models.

A first aspect of the invention provides the use of a compound of formula I:

RC(O)—NH—(CH₂)ₙ—CH═CH₂
wherein R represents C₁₋₂₀ alkyl, C₂₋₂₀ alkenyl or C₂₋₂₀ alkynyl; and
n is an integer from 0 to 3,
   in the manufacture of a medicament for use in pain relief.

A second aspect of the invention provides a compound of formula I as hereinbefore defined for use in medicine.

A third aspect of the invention provides a method of providing pain relief to a patient comprising administering to said patient an effective amount of a compound of formula I as hereinbefore defined.

Alkyl, alkenyl and alkynyl groups referred to herein may be straight or branched-chain. For the avoidance of doubt, alkenyl groups may contain one or (where appropriate) more carbon-carbon double bonds (eg 1 to 3 C═C double bonds). Moreover, alkynyl groups may contain one or (where appropriate) more carbon-carbon triple bonds (eg 1 to 3 C≡C triple bonds). Preferably alkenyl and alkynyl groups contain only one point of unsaturation. Also preferably the alkyl, alkenyl or alkynyl group is straightchained.

The compounds used in the invention are amides of fatty acids having in common a terminal allylamine head group.

The patient is a patient having, or likely to have, a need for pain relief. By ‘pain’ we include all types of pain, for example acute and chronic pains, such as neuropathic pain and post-operative pain, chronic lower back pain, cluster headaches, herpes neuralgia, post herpetic neuralgia, phantom limb pain, central pain, dental pain, opioid-resistant pain, cancer-related pain, visceral pain, surgical pain, bone injury pain, pain during labour and delivery, pain resulting from burns, including sunburn, post partum pain, migraine, angina pain and genitourinary tract-related pain including cystitis. The term includes nociceptive pain or nociception.

The patient may be any animal in need of pain relief, for example a human, horse, pig, cow, sheep, chicken, dog, cat, rat or a mouse. Preferably the patient is a human patient.

An embodiment of the first or third aspects of the invention is wherein the medicament further comprises one or more analgesics or the patient is administered said further one or more analgesics.

The further analgesic is preferably a cannabinoid receptor ligand, for example arachidonylethanolamide (AEA) or 2-arachidonylglycerol (2-AG) or palmitolyethanolamide (PEA), or a substrate for FAAH, for example PEA. Examples of other analgesics are set out below.

An embodiment of the first, second or third aspects of the invention is wherein, in the compound of formula I, R represents C₁₀₋₂₀ alkyl, C₁₀₋₂₀ alkenyl or C₁₀₋₂₀ alkynyl.

A further embodiment of the first, second or third aspects of the invention is wherein, in the compound of formula I, R represents C₁₀₋₂₀ n-alkyl, C₁₀₋₂₀ mono-alkenyl or C₁₀₋₂₀ mono-alkynyl.

A further embodiment of the first, second or third aspects of the invention is wherein, in the compound of formula I, R represents C₁₀₋₂₀ n-alklyl, C₁₀₋₂₀ mono-n-alkenyl or C₁₀₋₂₀ mono-n-alkynyl.

A further embodiment of the first, second or third aspects of the invention is wherein, in the compound of formula I, R represents C₁₁₋₁₉ n-alkyl, or C₁₁₋₁₉ mono-n-alkenyl.

A further embodiment of the first, second or third aspects of the invention is wherein, in the compound of formula I, R represents C₁₁₋₁₈ n-alkyl, or C₁₁₋₁₈ mono-n-alkenyl.

A further embodiment of the first, second or third aspects of the invention is wherein, in the compound of formula I, the alkenyl or alkynyl groups have no more than 3 C—C double or triple bonds, respectively.

A further embodiment of the first, second or third aspects of the invention is wherein the compound is not N-(2-propenyl)-5,8,11,14-eicosatetraenamide.

A further embodiment of the first, second or third aspects of the invention is wherein, in the compound of formula I, n represents 0 or 1, preferably n represents 1.

An embodiment of the first, second or third aspects of the invention is wherein the compound is N-(2-propenyl)hexadecanamide, N-(2-propenyl)cis-9-octadecenamide, N-(2-propenyl)cis-9-hexadecenamide, N-(2-propenyl)tetradecanamide, N-(2-propenyl)cis-9-tetradecenamide, N-(2-propenyl)octadecanamide, N-(2-propenyl)trans-9-octadecenamide, N-(2-propenyl)dodecanamide, or N-(2-propenyl)cis-5-dodecenamide. Preferably the compound is N-(2-propenyl)hexadecanamide. FIG. 1 includes illustrative diagrams of these compounds.

The compounds used in relation to the aspects of the invention may be prepared by any suitable method, as would be appreciated by a person skilled in the art.

For example:

N-(2-propenyl)hexadecanamide may be prepared from palmitoly chloride and allylamine as outlined in Scheme 1 of Vandevoorde et al (2003) J Med Chem 46 1440-1448.

N-(2-propenyl)cis-9-octadecenamide may be prepared from oleoyl chloride and allylamine. The compound is registered under CAS number 187529-39-1.

N-(2-propenyl)cis-9-hexadecenamide may be prepared from palmitoleic acid, oxalyl chloride and allylamine.

N-(2-propenyl)tetradecanamide may be prepared from myristoyl chloride and allylamine.

N-(2-propenyl)cis-9-tetradecenamide may be prepared from myristoleic acid, oxalyl chloride and allylamine.

N-(2-propenyl)octadecanamide may be prepared from stearoyl chloride and allylamine.

N-(2-propenyl)trans-9-octadecenamide may be prepared from elaidic acid, oxalyl chloride and allylamine.

N-(2-propenyl)dodecanamide may be prepared from lauroyl chloride and allylamine.

N-(2-propenyl)cis-5-dodecenamide may be prepared from cis-5-dodecenoic acid, oxalyl chloride and allylamine.

N-(2-propenyl)-5,8,11,14-eicosatetraenamide may be prepared from arachidonic acid, oxalyl chloride and allylamine. The compound is registered under CAS number 177037-49-9. The compound is mentioned in Boger et al (1999) Bioorg Med Chem Lett 9, 1151-1154; Lin et al (1998) J Med Chem 41, 5353-5361; Pate et al (1996) Life Sci 58, 1849-1860; WO 00/32200.

The reagents mentioned above can be purchased from Sigma-Aldrich-Fluka, Acros Chimica. If the acyl chlorides are not commercially available, they might be prepared from the corresponding carboxylic acids and oxalyl chloride in dichloromethane as will be appreciated by a person skilled in the art.

Provided below is a detailed protocol for the synthesis of N-(2-Propenyl)hexadecanamide:

In a two neck flask, with 5.7 g (100 mmol) of allylamine was poured into 10 mL of dry methylene chloride. The solution was cooled in an ice bath and magnetically stirred. 2.74 g (10 mmol) of palmitoyl chloride was added dropwise. The reaction mixture was stirred for 12 h at room temperature and then washed with 5% sodium bicarbonate solution, 1 M HCl, and brine. The organic layer was dried over MgSO4, and after filtration, the solvent was evaporated under reduced pressure to give 1.74 g (59%) of a white solid:

Physical and spectral data: mp 61-63° C. (uncorrected); TLC (ethyl acetate/methanol 8:2 vv-1) Rf) 0.77; 1H NMR (CDCl₃) % o (ppm) 0.87 (t, J) 3 Hz, 3H), 1.22-1.54 (m, 26H), 2.19 (t, J) 7 Hz, 2H), 3.7-3.72 (m, 2H), 5.11-5.2 (m, 2H), 5.55 (NH), 5.79-5.88 (m, 1H); ¹³C NMR (CDCl₃) % o (ppm) 14.10 (CH3), 22.77, 25.88, 29.43, 29.56, 29.76, 32.02, 35.39, 36.87, 41.99, 58.48, 116.39 (CH2), 134.51 (CH), 173.07 (C═O); mass spectrometry [M+•]) 296; IR Ó (cm-1) 3299 (NH), 1636 C═O). CAS number: 1012114-99-8.

The modifications required to synthesise further compounds used in the methods of the invention will be readily apparent to those of skill in the art.

The compounds of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

Preferably, the formulation is a unit dosage containing a daily dose or unit, daily sub-dose or an appropriate fraction thereof, of the active ingredient.

In human therapy, the compounds of the invention can be administered alone but will generally be administered in admixture with a suitable pharmaceutical excipient diluent or carrier selected with regard to the intended route of administration and standard pharmaceutical practice.

For example, the compounds of the invention can be administered orally, buccally or sublingually in the form of tablets, capsules, ovules, elixirs, solutions or suspensions, which may contain flavouring or colouring agents, for immediate-, delayed- or controlled-release applications. The compounds of invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The compounds of the invention can also be administered parenterally, for example, intravenously, intra-arterially, epidurally, intraperitoneally, intrathecally, intraventricularly, intrasternally, intracranially, intramuscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the compounds of the invention will usually be from 1 to 1000 mg per adult (i.e. from about 0.015 to 15 mg/kg), administered in single or divided doses.

Thus, for example, the tablets or capsules of the compound of the invention may contain from 1 mg to 1000 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The compounds of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™ or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains at least 1 mg of a compound of the invention for delivery to the patient. It will be appreciated that the overall daily dose with an aerosol will vary from patient to patient, and may be administered in a single dose or, more usually, in divided doses throughout the day.

Alternatively, the compounds of the invention can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route, particularly for treating diseases of the eye.

For ophthalmic use, the compounds of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Generally, in humans, oral or topical administration of the compounds of the invention is the preferred route, being the most convenient. In circumstances where the recipient suffers from a swallowing disorder or from impairment of drug absorption after oral administration, the drug may be administered parenterally, e.g. sublingually or buccally.

For veterinary use, a compound of the invention is administered as a suitably acceptable formulation in accordance with normal veterinary practice and the veterinary surgeon will determine the dosing regimen and route of administration which will be most appropriate for a particular animal.

Conveniently, the formulation is a pharmaceutical formulation. Salts of the compounds to be used in the invention may be prepared in conventional manner, for example by reaction of the compound with an appropriate base to form the corresponding base salt, or with an appropriate acid to form the corresponding acid salt. Physiologically acceptable acid salts include hydrochloride, sulphate, mesylate, besylate, phosphate and glutamate.

The compounds to be used in the invention may also be administered in the for of ‘prodrugs’.

The term “prodrug” as used in this application refers to a precursor or derivative form of a pharmaceutically active substance that is capable of being enzymatically activated or converted into the more active parent form (see, for example, D. E. V. Wilman “Prodrugs in Cancer Chemotherapy” Biochemical Society Transactions 14, 375-382 (615th Meeting, Belfast 1986) and V. J. Stella et al “Prodrugs: A Chemical Approach to Targeted Drug Delivery” Directed Drug Deliveiy R. Borchardt et al (ed.) pages 247-267 (Humana Press 1985)).

The prodrug may be, for example, easier to administer, more suitable for storage or less toxic or undesirable at the site of administration.

Several factors need to be taken into account in selecting an enzyme for prodrug activation. These include the molecular weight and physical properties of the enzyme, its activity and stability under physiological conditions, and the nature of the drug that the enzyme generates. The enzyme may be an endogenous enzyme or may be an exogenous enzyme targeted to a site where pain relief is required (for example a tumour site).

Enzymes of both mammalian and non-mammalian origin are currently being explored for the activation of a wide range of prodrugs (Senter et al, 1993. Generation of cytotoxic agents by targeted enzymes. Bioconjugate 4, 3-9; Senter et al, 1991. Activation of prodrugs by antibody-enzyme conjugates. In Immunobiology of Proteins and Peptides VI, ed. M. Z. Atassi. Plenum Press, New York, pp 97-105). While enzymes of mammalian origin might be advantageous due to reduced immunogenicity, the prodrugs that they act upon might be substrates for corresponding endogenous enzymes.

Enzymes that may be useful in the method of this invention include, but are not limited to alkaline phosphatase useful for converting phosphate-containing prodrugs into free drugs, arylsulphatase useful for converting sulphate-containing prodrugs into free drugs, proteases, such as serratia protease, thermolysin, subtilisin, carboxypeptidases and cathepsins (such as cathepsins B and L), that are useful for converting peptide-containing prodrugs into free drugs, D-alanylcarboxypeptidases, useful for converting prodrugs that contain D-amino acid substituents, carbohydrate-cleaving enzymes such as βgalactosidase and neuraminidase useful for converting glycosylated prodrugs into free drugs, β-lactamase useful for converting drugs derivatized with β-lactams into free drugs, and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as abzymes, can be used to convert the prodrugs of the invention into free active drugs [see, e.g. R J Massey, Nature, 328, pp. 457-458 (1987)].

Similarly, the prodrugs of this invention include, but are not limited to, the above-listed prodrugs, e.g., phosphate-containing prodrugs, thiophosphate-containing prodrugs, sulphate-containing prodrugs, peptide-containing prodrugs, D-amino acid-modified prodrugs, glycosylated prodrugs, P-lactam-containing prodrugs, optionally substituted phenoxyacetamide-containing prodrugs or optionally substituted phenylacetamide-containing prodrugs.

A fourth aspect of the invention is a pharmaceutical composition comprising a compound as defined in the first aspect of the invention and one or more analgesics and a pharmaceutically acceptable excipient.

A fifth aspect of the invention is a kit of parts comprising:
(a) a compound as defined in the first aspect of the invention; and,
(b) one or more analgesics; and,
(c) a pharmaceutically acceptable excipient

The carrier(s) must be “acceptable” in the sense of being compatible with the compound of the invention and not deleterious to the recipients thereof. Typically, the carriers will be water or saline which will be sterile and pyrogen free. Means of administration of a pharmaceutical composition are outlined above.

For example, the kit of parts may comprise a compound of formula I and one or more analgesics, wherein each are presented as a separate formulations and labelled for use together.

An embodiment of the first, third, fourth or fifth aspects of the invention is wherein the analgesic is an opioid, a non-steroidal anti-inflammatory drug, a local anaesthetic, a NMDA receptor antagonist, a cannabinoid, an antidepressant, and/or an anticonvulsant.

Examples of analgesics included in this embodiment of the aspects of the invention include opioid such as morphine, codeine, pethidine, methadone, aspirin and related compounds, ibuprofen, cyclooxygenase inhibitors such as acetaminophen (paracetamol), sodium channel blockers such as lidocaine, dibucaine and tetracaine, calcium channel bloackers, N-methyl-D-aspartate (NDMA) receptor antagonists such as ketamine and phencyclidine, cannabinoids such as anandamide (arachidonylethanolamide, AEA) and 2-arachidonylglyercol (2-AG), antidepressants such as the tricyclic antidepressants imipramine and the serotonin re-take inhibitor paroxetine, anti-convulsants such as gabapentin, carbamazepine and phenyloin and agents which interact with transient receptor potential (TRP) ion channels.

Any publications referred to herein are hereby incorporated by reference.

The invention will now be described in more detail by reference to the following non-limiting Figures and Examples.

FIG. 1:

(A) The structure of anandamide (AEA), 2-arachidonylglycerol (2-AG), palmitoylethanolamide (PEA) and palmitoylallylamide (L-29).

(B) The structure of compounds with similarities to PEA

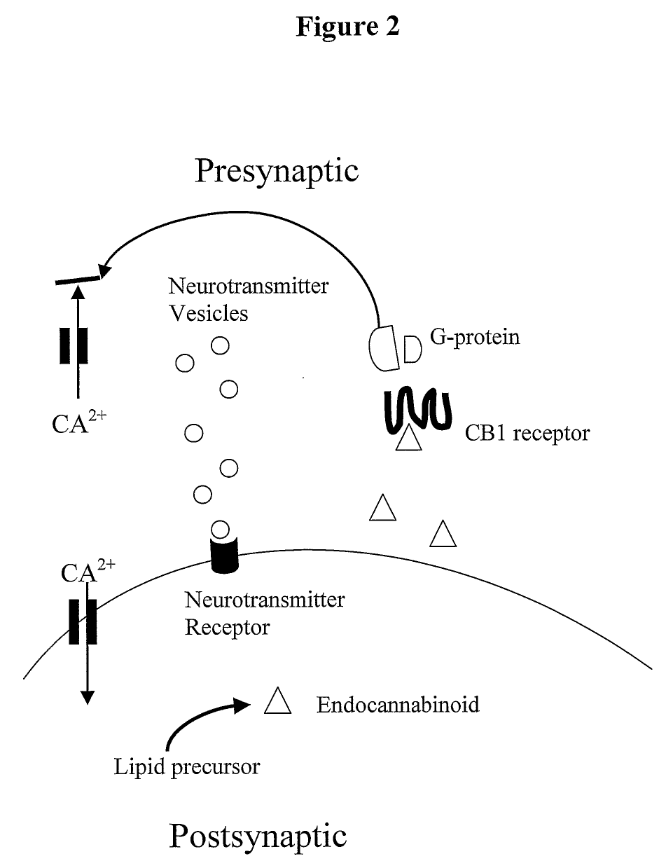

FIG. 2: Depolarisation-induced suppression of excitation

An action potential at the pre-synaptic cell leads to neurotransmitter release and binding to the post-synaptic receptor. Subsequent calcium influx causes endocannabinoid synthesis and release. Endocannabinoids travel in a retrograde fashion across the synapse and bind to the pre-synaptic CB₁ receptor. This activates G-proteins which block calcium channels, making it less likely that another action potential will reach threshold levels.

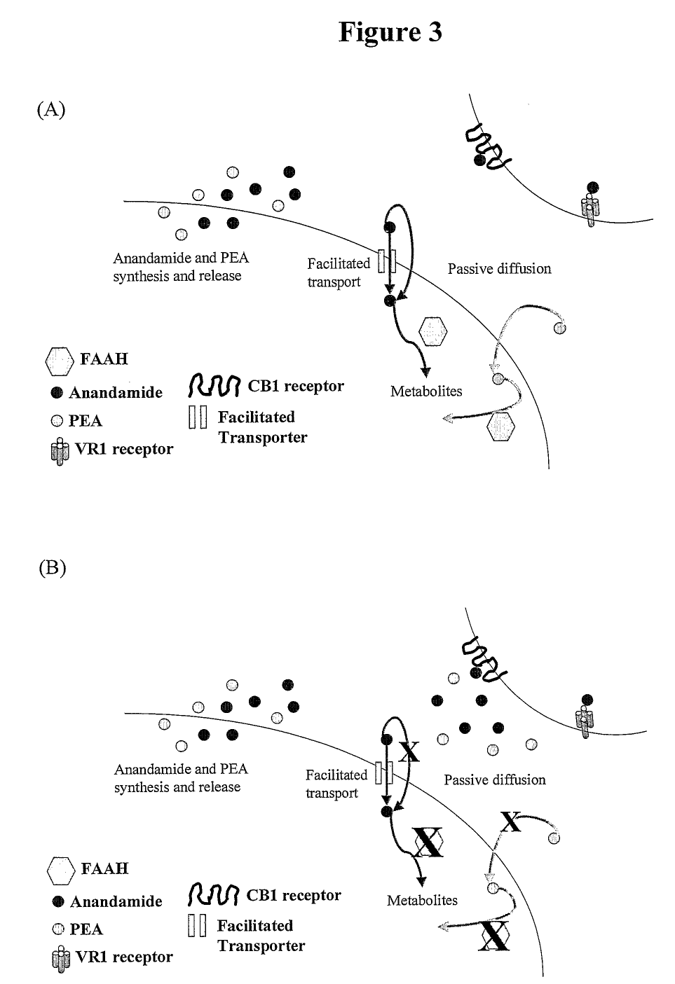

FIG. 3:

(A) Anandamide and other cannabinoids are synthesised and released into the synapse, where they can bind to the CB₁ receptor. AEA re-enters the cell via its concentration gradient by facilitated diffusion or passive diffusion. Once inside the cell endocannabinoids are broken down to inactive metabolites by FAAH.

(B) If FAAH is inhibited the concentration gradient into the cell is reduced and the amount of endocannabinoids present in the synapse increases, thereby increasing binding to CB₁ receptors

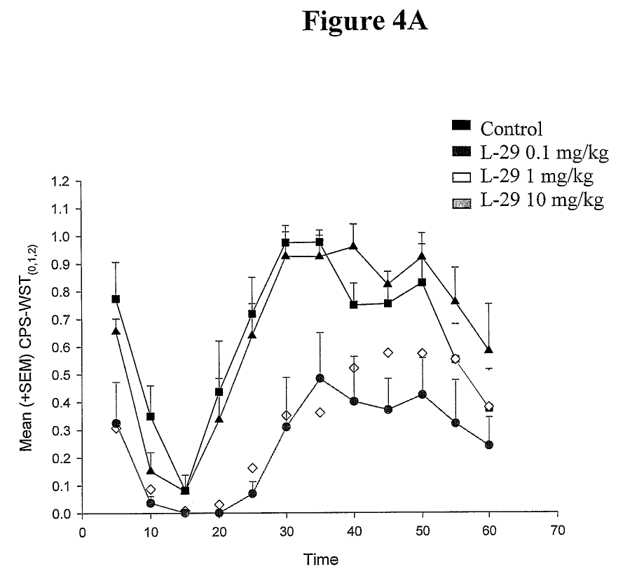

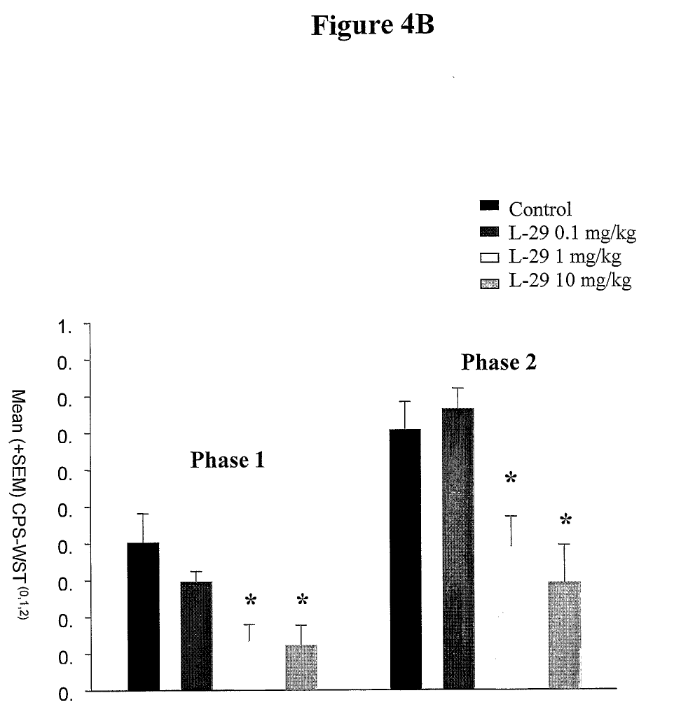

FIG. 4: Pain behaviour in the formalin test is attenuated by palmitoylallylamide.

(A) Time course of nociceptive activity (mean CPS-WST_{(0,1,2)}+S.E.M.) for the control group, 0.1 mg/kg L-29, 1 mg/kg L-29 and 10 mg/kg L-29, following s.c. injection of 2.5% formalin into the dorsal hind paw.

(B) Mean pain scores (mean CPS-WST_{(0,1,2)}+S.E.M.) for both phase 1 (0-15 min) and phase 2 (15-60 min) of the formalin test for the control group, 0.1 mg/kg L-29, 1 mg/kg L-29 and 10 mg/kg L-29.

*P<0.05 compared with control values by one-way ANOVA followed by Dunnett's test

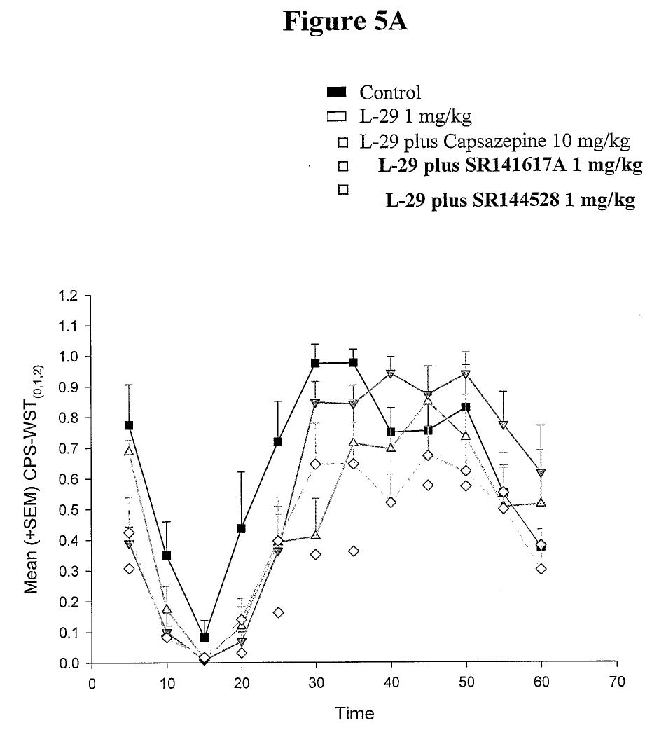

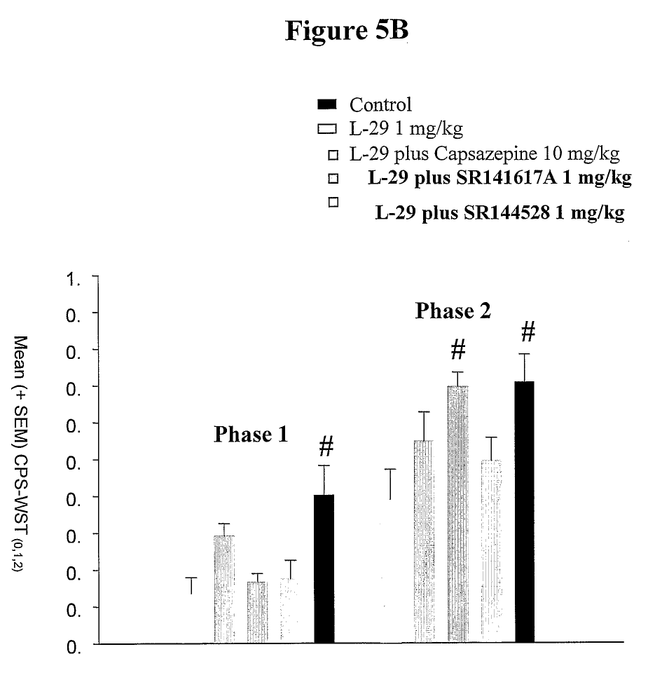

FIG. 5: The effect of receptor antagonists on palmitoylallylamide mediated antinociception.

(A) Time course of nociceptive activity (mean CPS-WST_{(0,1,2)}+S.E.M.) for the control group, 1 mg/kg L-29, and 1 mg/kg L-29 with prior administration of either 1 mg/kg SR141716A, 1 mg/kg SR144528 or 10 mg/kg capsazepine.

(B) Mean pain scores (mean CPS-WST_{(0,1,2)}+S.E.M.) for both phase 1 (0-15 min) and phase 2 (15-60 min) of the formalin test for the control group, 1 mg/kg L-29, and 1 mg/kg L-29 with prior administration of either 1 mg/kg SR141716A, 1 mg/kg SR144528 or 10 mg/kg capsazepine.

# P<0.05 compared with L-29 1 mg/kg values by one-way ANOVA followed by Dunnett's test

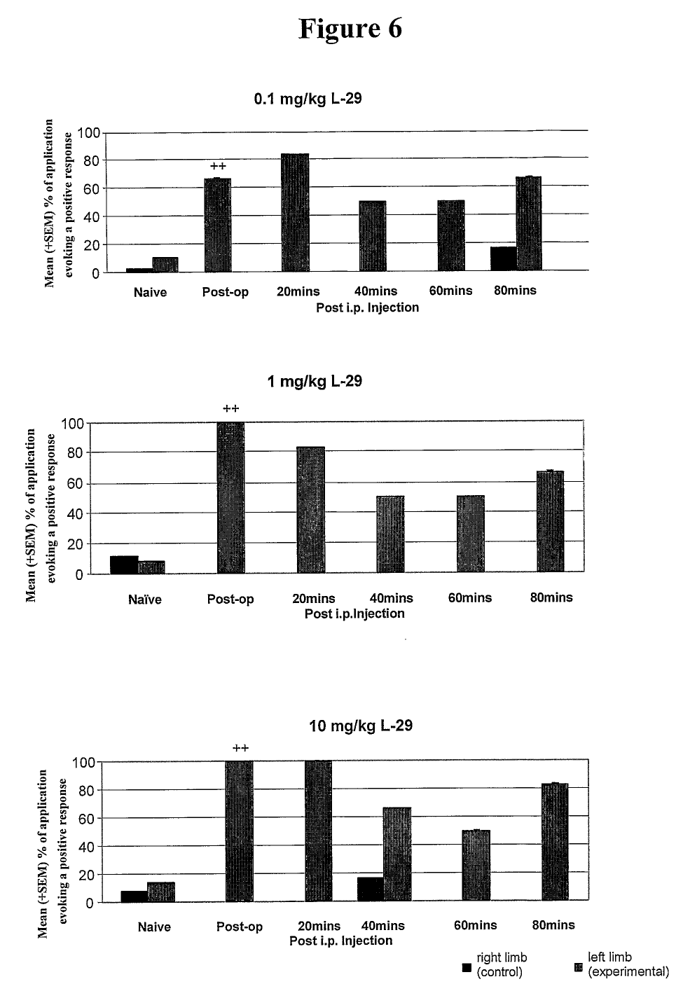

FIG. 6: Bilateral hind limb withdrawal responses to cold stimulation (acetone drop) in rats rendered neuropathic by partial sciatic nerve ligation. administered L-29 (0.1-10 mg/kg, i.p., n=6 per dose).

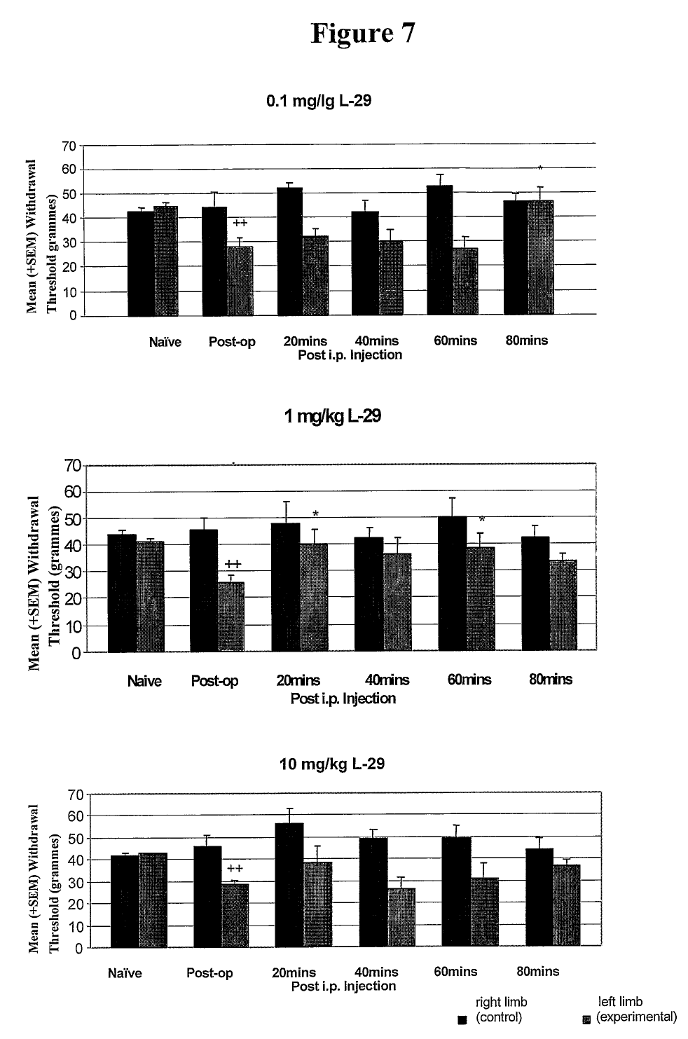

FIG. 7: Bilateral hind limb withdrawal responses to mechanical stimulation (electronic Von Frey) in rats rendered neuropathic by partial sciatic nerve ligation administered L-29 (0.1-10 mg/kg, i.p., n=6 per dose).

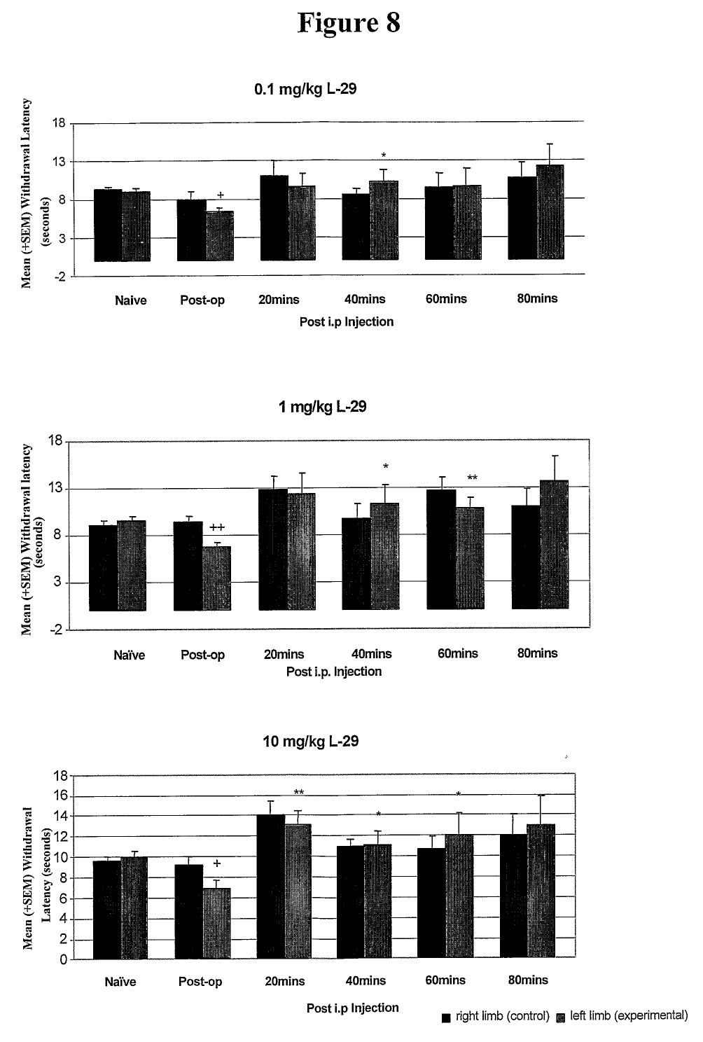

FIG. 8: Bilateral hind limb withdrawal responses to thermal stimulation (Hargreaves' device) in rats rendered neuropathic by partial sciatic nerve ligation administered L-29 (0.1-10 mg/kg, i.p., n=6 per dose).

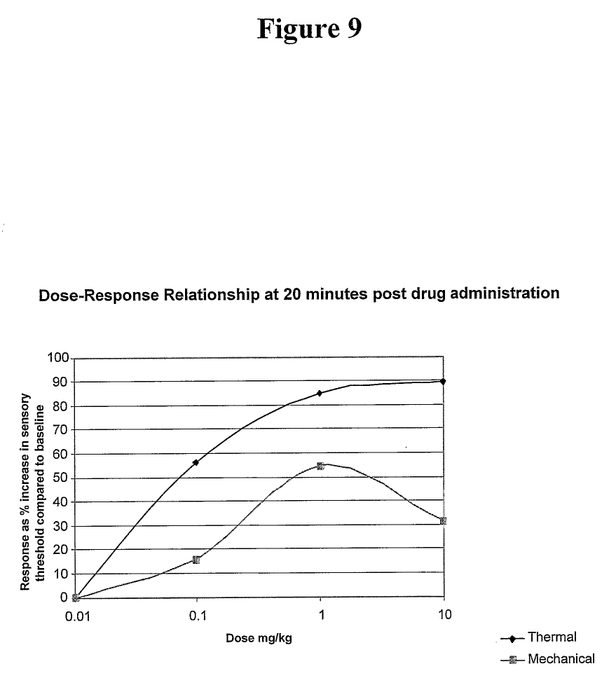

FIG. 9: Dose-response relationship for thermal and mechanical stimuli for L-29 (0.1-10 mg/kg) at 20 minutes post drug administration (where response is the % increase in sensory thresholds compared to baseline).

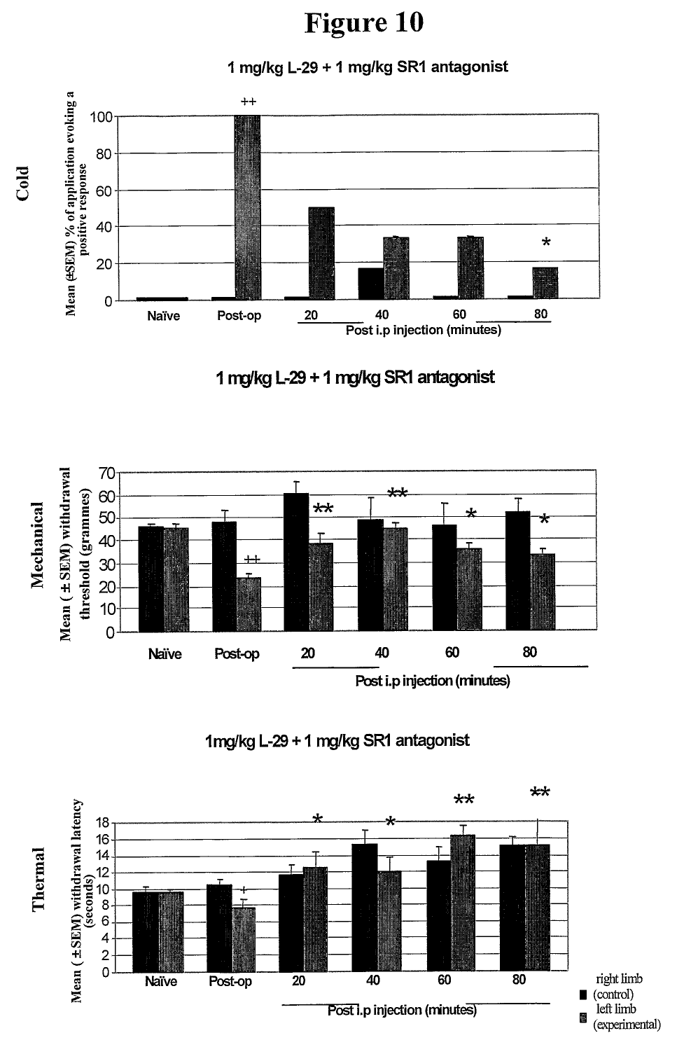

FIG. 10: Bilateral hind limb withdrawal responses to cold (acetone drop), mechanical (electronic Von Frey) and thermal (Hargreaves' device) stimulation in rats rendered neuropathic by partial sciatic nerve ligation (n==6 per group) co-administered SR141716a (1 mg/kg, i.p.) and an effective dose of L-29 (1 mg/kg, i.p.).

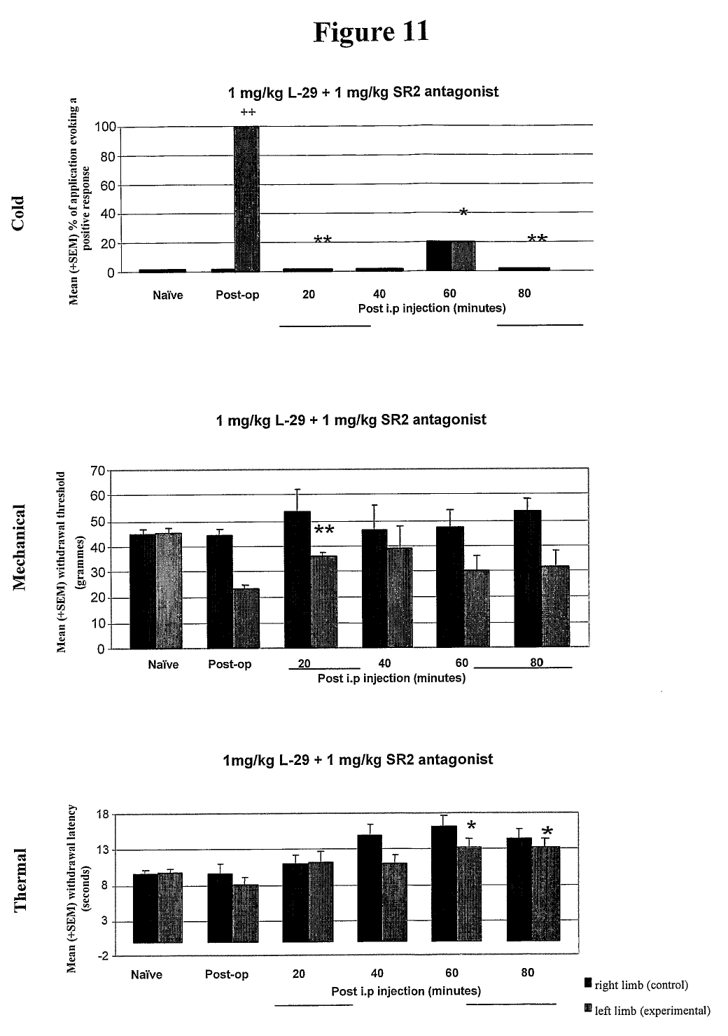

FIG. 11: Bilateral hind limb withdrawal responses to cold (acetone drop), mechanical (electronic Von Frey) and thermal (Hargreaves' device) stimulation in rats rendered neuropathic by partial sciatic nerve ligation (n=5 per group) co-administered SR144528 (1 mg/kg, i.p.) and an effective dose of L-29 (1 mg/kg, i.p.)

FIG. 12: Outline of Experimental Results

### EXAMPLE 1

### The Effect of Palmitoylallylamide, a Fatty Acid Amide Hydrolase Inhibitor, on Formalin-evoked Pain in the Rat

This study assessed the antinociceptive effect of a newly characterised fatty acid amide, palmitoylallylamide, in a rat model of inflammatory pain. Palmitoylallylamide is an analogue of palmitoylethanolamide, an endogenous cannabimimetic compound. Palmitoylallylamide inhibits fatty acid amide hydrolase but does not significantly bind to either CB₁ or CB₂ receptors. The therapeutic effects of palmitoylallylamide on the behavioural response to subcutaneous formalin injection were tested. Control animals displayed the characteristic biphasic (phase 1 and phase 2) response to formalin injection. Palmitoylallylamide (10 mg/kg and 1 mg/kg i.p.) significantly alleviated pain behaviour compared to solvent control in both phase 1 and phase 2. The selective CB₁ receptor antagonist, SR141716A (1 mg/lkg i.p.) significantly attenuated the antinociception produced by palmitoylallylamide in phase 2 of the formalin test. The selective CB₂ antagonist SR144528 (1 mg/kg i.p.) and the selective VR1 antagonist capsazepine (10 mg/kg i.p.) did not reverse palmitoylallylamide induced antinociception. These results support the hypothesis that fatty acid amide hydrolase inhibitors induce antinociception by increasing the extracellular levels of endocannabinoids and thereby increasing CB₁ receptor activation.

Abbreviations: L-29, Palmitoylallylamide; DMSO, Dimethyl sulphoxide; Δ⁹-THC, Δ⁹-tetrahydrocannabinol; PEA, Palmitoylethanolamide; SR1, SR141716A; SR2, SR144528; FAAH, Fatty acid amide hydrolase; i.p., Intraperitoneal; s.c., Subcutaneous; AEA, anandamide; 2-AG, 2-arachidonylglycerol; VR1, vanilloid receptor; CB₁, cannabinoid receptor 1; CB₂, cannabinoid receptor 2, WIN2, WIN 55, 212-2;

### 1. Introduction

Cannabis has been used for thousands of years to provide hemp fibre and for its psychotropic and analgesic effects, but it wasn't until the 1960's that meaningful research into cannabinoid compounds began. Since then the active constituents of cannabis have been described, synthetic cannabinoids have discovered and endogenous receptors and ligands identified.

The classical tetrad of cannabinoid effects are mediated by Δ⁹-tetrahydrocannabinol (Δ⁹-THC), the main psychoactive constituent of Cannabis sativa. These effects are analgesia, hypomotility, catalepsy and hypothermia. A major problem in cannabinoid research is how to separate the undesirable psychotropic effects of cannabinoids from their potential therapeutic benefits in pain, glaucoma, nausea and vomiting and spasticity. The discovery of mammalian cannabinoid receptors and endogenous ligands is allowing greater understanding of the mechanisms behind the physiological effects of cannabinoid compounds.

To date two cannabinoid receptors have been cloned, CB₁ in 1990 (Matsuda et al 1990) and CB₂ in 1993 (Munro et al 1993). CB₁ is expressed primarily on neurones. It has a very wide distribution in the brain, especially in pain processing areas (Egertova et al 1998) and is also found in the spinal cord (Farquhar-Smith et al 2000) and dorsal root ganglia. It appears to mediate most of the supraspinal effect of cannabinoids, as knockout mice (CB₁^{-/-}) do not display the central effects of Δ⁹-THC (Ledent et al 1999). CB₂ receptors were originally found in splenic macrophages and seem to be restricted to immune cell lines, although there have been reports that show CB₂ in brain microglia. Activation of CB₂ receptors appears to have an anti-inflammatory effect; there is some evidence this may be due to down-regulation of mast cells (Facci et al 1995).

Cannabinoids are unusual neurotransmitters as they are fatty acids, are synthesised ‘on demand’ instead of stored in the cell and usually travel in a retrograde fashion across the synapse. They appear to mediate the phenomenon of depolarisation-induced suppression of excitation (DSE) in the brain. Post-synaptic depolarisation activates the enzymes that synthesise endocannabinoids in the post-synaptic cell. The newly synthesised endocannabinoids then diffuse out of the post-synaptic cell and travel backwards across the synapse to bind to CB₁ receptors on the pre-synaptic cell membrane. Agonist binding to CB₁ receptors activates G-proteins that directly inhibit pre-synaptic Ca²⁺ entry into the pre-synaptic cell, thereby decreasing the probability of an action potential reaching threshold and neurotransmitter release (Krietzer and Regehr 2001) (FIG. 2). This may explain some of the analgesic properties of cannabinoids as they may disinhibit the descending pain-modulating pathways from the midbrain periaqueductal gray (PAG) and the rostral ventromedial medulla (RVM) both of which contain CB₁ receptors (Egertova et al 1998).

Fatty acid amide hydrolase (FAAH) is an enzyme that degrades many fatty acid amides including the endocannabinoids (Boger et al 2000a). It is distributed widely in the brain (Egertova et al 1998, Tsou et al 1998, Romero et al 2002) and periphery and is known to degrade both AEA and PEA (Tiger et al 2000). Recent evidence has shown that FAAH has complementary localisation with CB₁ receptors in many brain regions (Egertova et al 1998) including those involved in pain pathways. After endocannabinoids have been released from the synapse, they re-enter the post-synaptic cell by passive diffusion and facilitated transport along their concentration gradient. AEA is thought to travel mainly by facilitated transport (Day et al 2001, Jacobsson and Fowler 2001) whereas PEA transport is approximately 50% passive diffusion (Jacobsson and Fowler 2001) The two compounds may have different transporter molecules. Once inside the post-synaptic cell both compounds are metabolised by FAAH to inactive metabolites (FIG. 3A) (Tiger et al 2000).

Mice lacking the gene that encodes for the enzyme FAAH (FAAH^{-/-}) have been developed (Cravatt et al 2001) which have reduced pain sensation and increased levels of anandamide, evidence that FAAH regulates the level of endogenous cannabinoids. Inhibiting FAAH should increase the levels of AEA (Martin et al 2000, Day et al 2001, Deutsch et al 2001) and PEA inside the post-synaptic cell, thereby reducing the inward concentration gradient and therefore increasing the amount of endocannabinoids in the synapse. This will cause increased levels of CB₁ binding (FIG. 3B). The same applies to peripheral FAAH and increased extracellular levels of endocannabinoid. There is evidence that inhibiting FAAH causes cannabimimetic effects (Compton and Martin 1997), although the enzyme inhibitor used was not specific to FAAH.

Various FAAH inhibitors have been discovered (Martin et al 2000, Boger et al 2000b) and indeed PEA itself has some ability to inhibit FAAH (Jonsson et al 2001), evidence that lends weight to the entourage theory. The FAAH inhibitory compound that we are investigating is an analogue of PEA called palmitoylallylamide (L-29). It was developed by Dr Didier Lambert and team at the Unité de Chimie pharmaceutique et de Radiopharmacie, Universite catholique de Louvain, Brussels, Belgium. The capacity of L-29 to inhibit FAAH was calculated by measuring the ability of the compound to inhibit FAAH catalysed [³H]-AEA hydrolysis (maximum inhibition and pI50 calculated). L-29 achieved 67% of the maximum inhibition (±3%), so was reasonably efficacious, with a pI50 of 5.47 μM (±0.06), so was quite potent (unpublished data, but see (Jonsson et al 2001) for methods). In comparison, the values for PEA were 78% (±7%) and 5.3 μM (±0.15). Binding to CB₁ and CB₂ receptors was also calculated by measuring the displacement of radioactive [³H]-CP55,940 from a cell line transfected with CB₁ receptors and radioactive [³H]-WIN55,212-2 from a cell line transfected with CB₂ receptors. At a concentration of 10 μM, L-29 displaced 13.3% (±0.4%) in the CB₁ assay and 7.8% (±0.3%) in the CB₂ assay. For comparison PEA displaced 23.8% (±0.07%) and 13.9% (±1.7%) respectively, evidence that L-29 has a lower affinity at both the CB₁ and CB₂ receptors.

The formalin test was first described by Dubuisson and Dennis in 1977 (Dubuisson and Dennis 1977) and is a widely used and well-characterised model of acute and tonic inflammatory pain. Injection of dilute formalin into a rat hindpaw produces a biphasic pain-related behavioural response. There is an initial period of pain behaviour in the first five minutes after injection, followed by a period of quiescence lasting about ten to fifteen minutes. This is followed by a second period of pain behaviour, which continues until the end of the experiment.

Many schemes of scoring this pain behaviour have been proposed. Dubuisson and Dennis describe a weighted score that measures the amount of time spent in each of three behavioural categories; favouring the affected paw, elevating the affected paw and licking the affected paw (Dubuisson and Dennis 1977). Others have counted the number of flinches of the affected limb. Two papers have attempted to validate the different scoring methods: Abbott et al (1995) concluded that a simple sum of the time spent lifting and licking, or the scoring method of Dubuisson and Dennis were superior to any single measure and that adding flinching and favouring did not improve validity. Watson et al (1997) validated the optimal weighting method for the combined pain score and recommended alterations to the Dubuisson and Dennis method. Favouring is not scored, elevating the paw is given a weight of 1 and licking the paw is given a weight of 2. This new pain score was called the Combined Pain Score-Weighted Scores Technique (0,1,2) (CPS-WST_{(0,1,2)}) and is the method used in this study.

What are the mechanisms mediating the pain behaviour in the formalin test? It is generally believed that the first phase is due to direct chemical activation of primary afferent neurones of the C-fibre type (Puig and Sorkin 1995, Dallel et al 1995, Mc Call et al 1996). It seems that the C-fibres at the site of formalin injection are destroyed by the injection and it is those that are further from the injection site which receive a lower concentration of formalin that survive to respond to the stimulus (McCall et al 1996). Some authors believe that the second phase is due to the intense afferent barrage of the first phase causing central sensitisation in the dorsal horn neurones of the spinal cord (Martindale et al 2001). However, there have been numerous electrophysiological experiments showing continued C-fibre activation, albeit at a reduced intensity, throughout the second phase (Puig and Sorkin 1995, Dallel et al 1995); probably due to inflammation in the paw (Damas and Liegeois 1999). The most widely accepted hypothesis is that a combination of central sensitisation maintained by continuing primary afferent firing, due to ongoing inflammation produces pain behaviour in the second phase. There is also evidence that the interphase is due to descending inhibition from higher centres to the spinal cord (Puig and Sorkin 1995, Henry et al 1999) however others have reported less firing of C-fibres in the interphase (McCall et al 1996).

The aims of this experiment were firstly, to determine if L-29 is antinociceptive in the formalin model of inflammatory pain, and then to determine where this effect is mediated. To do this we used specific receptor antagonists prior to giving L-29 to see if they would reverse any possible antinociceptive effect. SR141716A (SR1) is a selective CB₁ receptor antagonist first described by (Rinaldi-Carmona et al 1995). Strangman et al (1998) described some of the pharmacokinetics of SR1. The authors administered WIN 55, 212-2 (WIN2), a potent CB₁ agonist, to rats inducing catalepsy. SR1 significantly antagonised WIN2 induced catalepsy within 15 min of administration (1 mg/kg i.p.), this effect lasted at least until the end of their experiments, approximately 50 minutes after administration. SR2 is a selective CB₂ receptor antagonist first described by (Ueda et al 2000). It has been used in studies at doses of 0.3 mg/kg and 3 mg/kg i.p. (Beaulieu et al 2000). Capsazepine is a competitive VR1 antagonist, developed by (Bevan et al 1991), and further described as antagonising the antinociceptive effect of capsaicin (Urban and Dray 1991, Dickenson and Dray 1991, Di Marzo et al 2001b). It has been used in studies at doses of 10 mg/kg i.p. (Bouaboula et al 1997). Therefore, we used SR1 (1 mg/kg), SR2 (1 mg/kg) and capsazepine (10 mg/kg) prior to L-29 administration.

### 2. Materials and Methods

### 2.1 Animals

All experiments conformed with British Home Office regulations. Male Wistar rats weighing 230-290 g were obtained from B&K. Animals were housed in group cages and maintained on a 12-hour light-dark cycle with ad libitum access to food and water. Each animal was used in one experiment only.

### 2.2 Materials

L-29 was a gift from D Lambert (see FIG. 1 for chemical structure). SR1 and SR2 were a gift from Sanofi and capsazepine was purchased from Tocris. All drugs were dissolved in 40% dimethyl sulphoxide (DMSO) and saline.

### 2.3 Treatment Groups

Rats were randomised into seven groups. Investigator JB carried out the dosing of the animals to conform with Home Office regulations, while investigator HJ carried out all other duties including scoring the behavioural test, she was blinded to the drugs given. All animals were acclimatised to the testing environment for at least 30 min prior to the experiment beginning. Each animal received a first intraperitoneal (i.p.) injection of either vehicle or antagonist and five minutes later a second i.p. injection of either vehicle or L-29. All i.p. injections had a volume of 1 ml/kg. 10 min later all the animals received a formalin injection. See Table 1 for groups.

### 2.4 Formalin Test

50 μl of 2.5% formalin was injected subcutaneously into the dorsum of the right hind paw via a 27G needle. The paw was then marked with permanent ink. The animal was immediately replaced in the observation chamber, a clear plexiglass box measuring 23 cm×18 cm×14 cm, with a mirror positioned underneath at 45° to allow an unobstructed view of the paw. Observations began as the rats were placed in the box and continued for 60 min. Their behaviour was rated according to the Composite Pain Score, Weighted Scores Technique (CPS-WST (0,1,2)), a validated pain behaviour scoring system for the rat formalin test (Watson et al 1997). The amount of time spent in each of 2 behavioural categories was measured for each consecutive 5 min period within the hour scored. Category 1, the injected paw is elevated and not in contact with any surface and category 2, the injected paw is licked, bitten or shaken. A weighted score was then obtained for each 5 min period and for phase 1 (0-15 min) and phase 2 (15-60 min), by using the formula below:

CPS-WST=(1×time in category 1)+(2×time spent in category 2)

### Test Session Duration

### 2.5 Statistical Analysis

Data was analysed using the one-way ANOVA test followed by Dunnett's test using the SigmaStat 2.3 computer package.

### 3. Results

### 3.1 Effects of L-29 on Formalin-Evoked Nociception

FIG. 4A shows the time course of pain behaviour following formalin injection for the control group and all doses of L-29 and FIG. 4B shows the mean pain score for phase 1 and phase 2 for these groups. The control group confirmed the previously described biphasic response to formalin (Dubuisson and Dennis 1977, Malmberg and Yaksh 1992, Abbott et al 1995) with an initial high pain score in the first 5 minutes, followed by a quiescent period and the beginning of the second phase, marked by increased pain scores which persist until the end of the experiment. Pain behaviour in both phases of the formalin test was dose-dependently inhibited by L-29 administration, but they maintained the biphasic pattern of pain behaviour. Both 10 mg/kg L-29 and 1 mg/kg L-29 significantly attenuated pain behaviour (P<0.05 by one-way ANOVA plus Dunnett's test v control), but 0.1 mg/kg L-29 was not significantly different from control. Although the 10 mg/kg dose of L-29 was not significantly more antinociceptive than the 1 mg/kg dose, a trend can be seen in FIG. 4A.

3.2 Effects of receptor antagonists on L-29-evoked analgesia

FIG. 5A shows the time course of pain behaviour for the control group, 1 mg/kg L-29 group and the receptor antagonist groups, which were 1 mg/kg L-29 with prior administration of either 1 mg/kg SR1, 1 mg/kg SR2 or 10 mg/kg capsazepine. FIG. 5B shows the mean pain scores for phase 1 and phase 2 of the above groups. All groups show the biphasic response to formalin injection. We wanted to discover whether the receptor antagonists decreased the antinociceptive effect of the L-29, so we analysed the data against the L-29 group. In phase 1 of the formalin test, the only group that was significantly different (P<0.05 by one-way ANOVA with Dunnett's test v 1 mg/kg L-29) to L-29 was the control group. In phase 2 however, SR1 reverses the antinociceptive effect of L-29 in phase 2 of the formalin test. SR2 and capsazepine do not significantly reverse the antinociceptive effect of L-29 in either phase.

### 4. Discussion

The results demonstrate that L-29 reduces pain behaviour in a dose-dependent manner in both phase 1 and phase 2 of the formalin model of inflammatory pain in the rat. SR1, a selective CB 1 receptor antagonist, significantly prevents this antinociceptive effect of L-29 in phase 2, whereas SR2 and capsazepine do not reverse this action. This suggests that the effects of L-29 are mediated by increased levels of endocannabinoids binding to CB 1 receptors. Surprisingly, although both phase 1 and phase 2 nociceptive behaviour was attenuated by L-29, only the antinociceptive effect in phase 2 was reversed by SR1.

In all animal behavioural tests environmental factors have a large influence, if not carefully controlled this can lead to bias. Novelty stress induced analgesia is a recognised phenomenon in rats placed in new environments, mediated by release of endogenous opiates (review Yamada and Nabeshima 1995). To minimise any such variation we kept to a strict protocol. All rats were 6-8 weeks old and arrived from the supplier at the animal house at least 3 days before the experiment started. They were housed in group cages of 5 as rats become stressed when housed in isolation, and were handled regularly to reduce stress due to handling during the experiment. On the day of the experiment rats arrived at the lab at least 1 hour before experiments began and were acclimatised to the observation chamber for at least 30 min before starting the experiment. There is also evidence that ambient temperature can affect the formalin test (Rosland 1991), so all experiments were carried out in the same climate controlled room. Site of injection can also lead to variation (Puig and Sorkin 1995), so all injections were given by same investigator and any rats without noticeable oedema and redness in the injected paw (indicating failure of the injection) were excluded.

The protocol for administering the treatment drugs was decided by reference to previous work. Pharmacological data available for SR1, (Strangman et al 1998) showed that SR1 has a latency to effect of approximately 15 min, so we decided to administer all antagonist drugs 15 min before starting experiment. Previous data is not available for L-29, so we based our decision on available PEA data, which is structurally similar to L-29. PEA has been given i.p. immediately prior to the formalin test (Jaggar et al 1998b) and has also been given i.p. 30 minutes prior to administration of noxious substances in the writhing test (Calignano et al 2001). We therefore decided to give L-29 10 minutes before formalin administration, as agonists should generally be given after their antagonist. This is because an antagonist may not work to fall effect if the agonist is already occupying the receptor site. Dose of formalin used is also important (Lee et al 2000). Lower concentrations will produce smaller pain responses, potentially making it difficult to detect an analgesic effect. If too high a concentration is used, the maximum pain behaviour level may be reached, making it difficult to detect a hyperalgesic effect. We used a median dose of 2.5% formalin, which has been used routinely in our laboratory.

There are many different animal models of pain. Differences between nociceptive assays include the stimulus aetiology, intensity, location and duration of the stimulus and the characteristics of the response. The formalin test is caused by a chemical/inflammatory stimulus given in a subcutaneous location producing a moderate intensity stimulus with a tonic but limited duration. It produces an organised and integrated behavioural response, not simply a reflex action. It's main advantage over models that look at simple nociceptive stimuli, such as the tail-flick test, are that it mimics human clinical pain conditions, in which pain lasts for a longer period of time and is not escapable. The immediate onset and limited duration are an advantage over the more prolonged time course of other inflammatory pain models, such as carrageenan. Another advantage is that the formalin test uses a freely moving animal which prevents the confounding factor of endogenous analgesia caused by the stress of restraint. A disadvantage of the formalin test is that the biphasic nature of the response makes interpretation of results more difficult when pharmacokinetics are uncertain. Although the formalin test is a robust model of inflammatory pain, further work using different pain models is needed to confirm the results we obtained in this study.

SR1 and SR2 may be inverse agonists rather than pure antagonists at cannabinoid receptors (Bouaboula et al 1997). Inverse agonists activate the receptor to produce effects opposite to that of the agonist. There is evidence that SR1 may cause hyperalgesia at the CB₁ receptor, suggesting either antagonism of an endogenous cannabinoid tone or an inverse agonist effect. Calignano et al 1998 showed a hyperalgesic effect of SR1 in the formalin test, however others (Beaulieu et al 2000) have not replicated this effect. Studies demonstrating an increase in levels of endogenous cannabinoids following formalin injection (Walker et al 1999) provide evidence that SR1 causes a reversal of endogenous tone rather than an inverse agonist effect. There is also extensive evidence that SR1 has no effect in many other systems (for review see Martin and Lichtman 1998).

The VR1 receptor is a cation channel activated by noxious heat and capsaicin, the pungent ingredient in chilli peppers, and mediates ‘burning’ pain sensation. AEA is an endogenous ligand of cannabinoid receptors, and there is also evidence that it is a full agonist at VR1 (Smart et al 2000), although in this study this action was not at physiological relevant concentrations. On the other hand, there is some evidence that the action of anandamide at VR1 receptors can be potentiated in certain circumstances. Inhibiting hydrolysis of anandamide has been shown to enhance the potency of anandamide as a VR1 ligand by at least 5 times (De Petrocellis et al 2001a). Increased levels of PEA have also been shown to increase anandamide action at VR1 receptors (De Petrocellis et al 2001). Therefore, in view of a possible effect at VR1 receptors, we used capsazepine, a potent and selective VR1 antagonist (Bevan et al 1992) to detect any possible effect.

FAAH has a wide substrate specificity and is capable of metabolising a wide range of AEA analogues and other fatty acid amides such as PEA and oleamide. Several standard compounds are capable of blocking FAAH activity including phenylmethylsulfonyl fluoride (PMSF) (Compton and Martin 1997) and methylarachidonylfluorophosphonate (MAPH) (Martin et al 2000). However these cannot be used therapeutically as they are too toxic, so various AEA analogues and other fatty acid derivatives have been investigated for their ability to inhibit FAAH (Lambert et al 1999, Boger et al 2000). It is doubtful whether FAAH inhibition will have much analgesic effect in the normal state, as many FAAH inhibitors lack cannabimimetic effects in vivo, although mice lacking FAAH (FAAH−/−) do have reduced pain sensation (Cravatt et al 2001). FAAH inhibitors may well be more beneficial in inflammatory states as levels of AEA and PEA are increased in these circumstances, for instance in a mouse model of multiple sclerosis (Baker et al 2000) and increased brain levels have been detected in response to the formalin test (Walker et al 1999). In addition, AEA and PEA given exogenously have been found to reduce inflammatory pain (Jaggar et al 1998a, Calignano et al 2001).

This study has demonstrated that the FAAH inhibitor L-29 may work primarily to increase the levels of endogenous cannabinoids available for binding to CB₁ receptors, however further work is needed to confirm our results and extend the understanding of the mechanisms of action of L-29. Another possibility could be that L-29 produces analgesic effects due to actions on a novel non-CB₁/CB₂ receptor. It is widely believed that there is a novel cannabinoid receptor, but this hypothesis has not yet been proved by cloning of the receptor. The most likely endogenous ligand for this putative receptor is PEA. This makes it less likely that L-29 is acting via this receptor, as PEA's effects are antagonised by CB₂ receptor antagonists, not CB₁ receptor antagonists. It would also be useful to confirm the action of L-29 in several diverse pain models. A model of reflex withdrawal such as the tail-flick test (D'Amour and Smith 1941); a model of visceral pain, such as colorectal distension (Ness and Gebhart 1988) or inflammatory visceral pain produced by turpentine instillation into the urinary bladder (McMahon and Abel 1987) and perhaps a model of neuropathic pain such as L5 and L6 spinal nerve ligation (Kim and Chung 1992). Tests performed after L-29 administration to evaluate the tetrad of cannabimimetic effects: catalepsy, hypolocomotion, analgesia (already done) and hypothermia would determine if there is a generalised rise in endogenous cannabinoids. Anandamide produces all these cannabimimetic effects when given exogenously, so if the main effect of L-29 is to increase endogenous anandamide, as suggested by administration of SR1, then L-29 may also produce similar effects.

CB1 agonists are undoubtedly antinociceptive, both in a variety of animal models of pain (review Pertwee 2001) and in clinical pain states, but dose-limiting psychotropic side effects have restricted their use thus far. Therefore new strategies for manipulating the endocannabinoid system are being developed (review Porter and Felder 2001). Partial agonists, receptor modulators which have no agonist activity but potentiate the response to agonists, reuptake inhibitors and FAAH inhibitors may have more potential as therapeutic agents. In this study we have demonstrated an antinociceptive role for a FAAH inhibitor in a clinically relevant model of inflammatory pain.

### REFERENCES

Abboft et al Pain 60 (1995) 91-102 Baker et al FASEB Journal 15 (2001) 300-2
Beaulieu et al European Journal of Pharmacology 396 (2000) 85-92
Bevan et al Br. J. Pharmacol. 102 (1991) 77
Boger et al Bioorganic & Medicinal Chemistry Letters 10 (2000) 2613-2616
Boger et al PNAS 97 (2002) 5044-5049
Bouaboula et al The Journal of Biological Chemistry 272 (1997) 22330-22339
Calignano et al Nature 394 (1998) 277-281
Calignano et al European Journal of Pharmacology 419 (2001) 191-198
Compton and Martin Journal of Pharmacology and Experimental Therapeutics 283 (1997) 1138-1143
Cravatt et al PNAS 98 (2001) 9371-9376
Dallel et al Pain 61 (1995) 11-16
Damas and Liégeois Naunyn-Schmiedeberg's Arch. Pharmacol. 359 (1999) 220-227 'D'Arnour and Smith J. Pharmacol. Exp. Ther. 72 (1941) 72
Day et al Molecular Pharmacology 59 (2001) 1369-1375
De Petrocellis et al FEBS Letters 506 (2001) 253-256
Deutsch et al Journal of Biological Chemistry 276 (2001) 6967-6973
Dickenson and Dray Br. J. Pharmacol. 104 (1991) 1045-9
Di Marzo et al TRENDS in Pharmacological Sciences 22 (2001) 346-349
Di Marzo et al Eur. J. Pharmacol. 420 (2001) 123-31
Dubuisson and Dennis Pain 4 (1977) 161-174
Egertova et al Proc. R. Soc. Lond. 265 (1998) 2081-2085
Facci et al Proc, Natl. Acad. Sci. USA 92 (1995) 3376-3380
Farquhar-Smith et al Molecular and Cellular Neuroscience 15 (2000) 510-521
Henry et al Pain 82 (1999) 57-63
Hillard Prostaglandins Other Lipid Mediat. 61 2000 3-18
Jacobsson and Fowler British Journal of Pharmacology 132 (2001) 1743-1754
Jaggar et al Pain 76 (1998) 189-199
Jaggar et al Neuroscience Letters 253 (1998) 123-126
Jonsson et al British Journal of Pharmacology 133 (2001) 1263-1275
Kim and Chung Pain 50 (1992) 355
Kreitzer and Regehr Neuron 29 (2001) 717-727
Lee et al Acta Anaesthesiol. Sin. 38 (2000) 59-64
Ledent et al Science 283 (1999) 401-404
Malmberg and Yaksh The Journal of Pharmacology and Experimental Therapeutics 263 (1992) 136-146
Martin et al Journal of Pharmacology and Experimental Therapeutics 294 (2000) 1209-1218
Martin and Lichtman Neurobiol. Dis. 5 (1998) 447-461
Martindale et al Neuroscience Letters 316 (2001) 33-36
Matsuda et al Nature 346 (1990) 561-564
McCall et al Neuroscience Letters 208 (1996) 45-48
McMahon and Abel Pain 28 (1987) 109-127
Munro et al Nature 365 (1993) 61-65
Ness and Gebhart Brain Res. 450 (1998) 153
Pertwee Progress in Neurobiology 63 (2001) 569-611
Porter and Felder Pharmacology and Therapeutics 90 (2001) 45-60
Puig and Sorkin Pain 64 (1995) 345-355
Rinaldi-Carmona et al Journal of Pharmacology and Experimental Therapeutics 284 (1998) 644-650
Rinaldi-Carmona et al Life Sciences 56 (1995) 1941-1947
Romero et al Molecular Brain Research In Press
Rosland Pain 45 (1991) 211-6
Smart et al Br. J. Pharmacol. 129 (2000) 227-30
Strangman et al Brain Research 813 (1998) 323-328
Tiger et al Biochemical Pharmacology 59 (2000) 647-653
Tsou et al Neuroscience Letters 254 (1998) 137-140
Ueda et al Chemistry and Physics of Lipids 108 (2000) 107-121
Urban and Dray Neurosci. Lett. 134 (1991) 9-11
Walker at al PNAS 96 (1999) 12198-12203
Watson et al Pain 70 (1997) 53-58
Yamada and Nabeshima Behav. Brain. Res. 67 (1995) 133-45

### APPENDIX

TABLE 1

| Group | First i.p Injection | | Second i.p Injection | |
|---|---|---|---|---|
| 1 | Vehicle | | Vehicle | |
| 2 | Vehicle | | 10 | mg/kg L-29 |
| 3 | Vehicle | | 1 | mg/kg L-29 |
| 4 | Vehicle | | 0.1 | mg/kg L-29 |
| 5 | 1 | mg/kg SR144528 | 1 | mg/kg L-29 |
| 6 | 1 | mg/kg SR141716A | 1 | mg/kg L-29 |
| 7 | 10 | mg/kg capsazepine | 1 | mg/kg L-29 |

### EXAMPLE 2

### Assessment of the Antinociceptive Effect of Palmitoylallylamide (L-29) in a Rat Model of Neuropathic Pain

### 1. Introduction

Palmitoylallylamide (L-29) is a structural analogue of the endogenous cannabimimetic compound palmitoylethanolamide (PEA), but does not significantly bind to either CB₁ or CB₂ receptors. L-29 inhibits fatty acid amide hydrolase, the enzyme known to degrade endogenous cannabinoids including the prototypical ligands anandamide and PEA. This mechanism is thought to induce antinociception by increasing the extracellular levels of endocannabinoids and thereby increasing CB₁ receptor activation. It is also possible that L-29 acts via an uncharacterised CB₂-like receptor.

The therapeutic effect of L-29 on the behavioural response in a rat model of formalin-evoked inflammatory pain has previously been investigated.

However, its effect in an established rodent model of neuropathic pain has yet to be studied. There were two aims: firstly, to assess the antinociceptive effect of L-29 in a partial sciatic nerve ligation model as previously described by Seltzer et al.,² and then determine where this effect is mediated. To establish this, we will utilise the selective CB₁ and CB₂ receptor antagonists, SR1 (SR141716a) and SR2 (SR144528).

### 2. Specific Aims

Reproduce an established rat model of partial sciatic nerve ligation (PSL) as previously described by Seltzer et al.² as a model of peripheral nerve injury related neuropathy.

Investigate whether rats in this model display behavioural features of neuropathic pain and whether behavioural response is altered by L-29.
Identify through which cannabinoid receptor this antinociceptive effect may be mediated by using selective CB1 and CB2 receptor antagonists.
Dose/response curves were constructed.

### 3. Materials and Methods

All experiments were performed in accordance with British Home Office regulations over a five week period who were blinded to the drugs being administered.

### 3.1 Animal Maintenance

Male Wistar rats, weighing 250-350 g (mean 300 g) were housed at a constant temperature under a 14:10 light/dark cycle, with free access to food and water.

3.2 Experimental Groups

The animals were randomised into four experimental groups, with six animals in each group:

### Group A. Solvent Control (DMSO Vehicle+Saline)

Behavioural thresholds to all three modalities were recorded at time (t)=0. Each animal then received 1 ml total volume intraperitoneal (i.p.) injection of 40% dimethyl sulphoxide (DMSO) vehicle and saline. No drug was administered.

Group B. L-29 in a DMSO Vehicle in Three i.p. Doses: 0.1 mgkg⁻¹, 1 mgkg⁻¹, 10 mgkg⁻¹

Previous experiments in a model of formalin-evoked inflammatory pain¹ investigated three dose groups of L-29 (0.1 mgkg⁻¹, 1 mgkg⁻¹ and 10 mgkg⁻¹ administered i.p.) and demonstrated that while both 1 mgkg⁻¹ and 10 mgkg⁻¹ doses significantly attenuated pain behaviour, an i.p. dose of 0.1 mgkg⁻¹, L-29 was not significantly different from the control group. In this study, six animals were allocated to each dose group and baseline limb withdrawal thresholds were recorded for each animal at t=0. This was immediately followed by the ip. administration of L-29 solution.

Group C. CB₁ receptor antagonist: SR1 (1 mgkg⁻¹ i.p.)

Limb withdrawal thresholds to all three modalities were recorded at t=0. Each animal then received i.p. injection of the antagonist in a 40% DMSO and saline solution, immediately followed by the administration of L-29 at a dose of 1 mgkg⁻¹ via the same route. Sensory testing resumed and limb withdrawal thresholds were recorded over the course of the experiment as described.

Group D. CB₂ Receptor Antagonist: SR2 (1 mgkg⁻¹ i.p.)

As for group C.

### 3.3 Partial Sciatic Nerve Injury) (PSL Model)

Following the original description for partial sciatic nerve ligation by Seltzer et al.², surgery was performed in equal number. Under inhalational anaesthesia using isoflurane and nitrous oxide, the left sciatic nerve was exposed at upper-thigh level and the dorsal third—half of the sciatic nerve tightly ligated with a 7-0 silk suture at a site just distal to the point at which the posterior biceps semitendinosus nerve branches off the sciatic nerve. Haemostasis was confirmed and the wound then closed with muscle and skin sutures and a small volume of 0.5% bupivicaine administered subcutaneously. A strict aseptic technique was maintained throughout. The animal was then recovered and returned to its housing environment and a post-operative check conducted the following day. See FIG. 12 for full description and protocol.

TABLE 1B
Outline of Experimental Procedure
On day of surgery, acclimatise rat for 15 mins and measure pre-op
baseline sensory thresholds to cold, mechanical and thermal stimuli (as per
protocol)
Perform partial sciatic nerve ligation surgery (as per protocol)
On day 8 post-op, repeat behavioural testing (as above) to confirm
neuropathy
Measure sensory thresholds for all modalities at t = 0.
Administer i.p. injection of solvent control or L-29 solution (i.p.
antagonist solution will precede the administration of L-29 solution)
Measure sensory thresholds to each modality 20, 40, 60 and 80 minutes
post drug administration

### 3.4 Sensory Testing

Behavioural tests included simple reflex withdrawal to mechanical, thermal and cold stimuli. Before commencing experiments, animals were allowed to acclimatise to the testing environment for at least 15 minutes until exploratory behaviour had ceased. In order to minimise bias, the experiment to be conducted was randomised (according to a predetermined random number table—see table 2) on the day of testing and the drug doses were drawn up and given by a separate investigator to the one scoring the behavioural tests who was blinded to the drugs given. Baseline sensory thresholds were measured pre-operatively on the day of surgery and on post-operative day eight. All animals included in drug testing displayed a statistically significant lowering of sensory thresholds (p<0.05) compared to the pre-operative thresholds in all sensory modalities. Sensory thresholds were recorded at time (t) 0, 20, 40, 60 and 80 minutes post drug administration.

TABLE 2
Randomisation table

| | |
|---|---|
| 1. | control |
| 2. | SR2 |
| 3. | L-29 (1) |
| 4. | SR1 |
| 5. | L-29 (10) |
| 6. | sham |
| 7. | sham |
| 8. | SR1 |
| 9. | L-29 (10) |
| 10. | SR2 |
| 11. | control |
| 12. | L-29 (1) |
| 13. | SR2 |
| 14. | SR1 |
| 15. | sham |
| 16. | L-29 (10) |
| 17. | L-29 (1) |
| 18. | control |
| 19. | SR1 |
| 20. | L-29 (1) |
| 21. | sham |
| 22. | control |
| 23. | L-29 (10) |
| 24. | SR2 |
| 25. | L-29 (10) |
| 26. | SR2 |
| 27. | control |
| 28. | L-29 (1) |
| 29. | SR1 |
| 30. | sham |
| 31. | control |
| 32. | SR1 |
| 33. | SR2 |
| 34. | sham |
| 35. | L-29 (1) |
| 36. | L-29 (10) |

### 3.4.1 Cold Allodynia

Cold allodynia was assessed using the acetone application technique, modified from Carlton et al.³. Animals were placed in a clear Plexiglas box (23×18×14 cm) with a 0.8 cm plastic mesh flooring. A single bubble of acetone was carefully applied to the mid-plantar surface of each hind paw and the animal's response noted. A response was taken as positive if there was paw withdrawal accompanied by a pain response, e.g. non-weight bearing, nuzzling of paw or vocalisation. Each paw was sampled five times, with at least three minutes between each test and a mean % positive withdrawal response calculated.

### 3.4.2 Mechanical Allodynia

Bilateral hind limb withdrawal thresholds to noxious mechanical stimuli were used to assess mechanical allodynia. Sampling was conducted using a calibrated electronic von Frey device (0.5 mm diameter force transducer tip) applied manually at a constant rate (5-8 g/sec) to the mid-plantar surface of the hind paw. Mean withdrawal thresholds were taken from a set of five applications, not less than ten seconds apart.

### 3.4.3 Thermal Hyperalgesia

Thermal hyperalgesia is assessed using a noxious infrared heat stimulus applied to the plantar surface of both hind paws as described by Hargreaves et al.⁴ Paw withdrawal thresholds to a focused beam of radiant heat at a constant temperature of 46° C. and infrared intensity (of twenty for rats and thirty for mice) is measured. A standard cut-off latency of 21.4 seconds is used to prevent possible tissue damage. Sampling is repeated five times to each paw with three minutes between testing and a mean withdrawal threshold is calculated.

### 3.5 Data Analysis

Statistical significance was determined for neuropathy by the paired t-test and for drug effects by the one-way ANOVA (Dunnett's test) (compared to post-operative values), both taking p<0.05 as statistically significant.

### 4. Results

Thirty-five animals demonstrated altered sensory thresholds in all modalities 8 days following partial sciatic nerve ligation surgery. This was confirmed by statistical analysis using the paired t-test (+p<0.05, ++p<0.005). Only one animal was excluded from the study as it exhibited a complete denervation in the operated limb, with no withdrawal response to any sensory modality tested.

In those animals in which solvent (40% DMSO and saline) was administered as a control following surgery, there was no significant difference from baseline sensory thresholds. This was confirmed by statistical analysis using the one-way ANOVA (Dunnett's) (*P<0.05, **p<0.005)

### 4.1 L-29 Studies

### 4.1.1 Thermal Hyperalgesia

Attenuation of thermal hyperalgesia was greatest at a dose of 10 mgkg⁻¹ between 20 and 60 minutes post i.p. drug administration. At a dose of 1 mgkg⁻¹, thermal hyperalgesia was attenuated later between 40 and 60 minutes post drug injection and only at 40 minutes at a dose of 0.1 mgkg⁻¹ L-29. (FIG. 8).

### 4.1.2 Mechanical Allodynia

A significant increase in mechanical sensory thresholds and hence reversal of the mechanical allodynia produced by partial sciatic nerve ligation, was observed at a dose of 1 mgkg⁻¹ L-29 at 20 minutes and 60 minutes post i.p. drug administration. No effect on mechanical allodynia was observed at a dose of 10 mgkg⁻¹ and only at 80 minutes post i.p drug administration for 0.1 mgkg⁻¹ (FIG. 7).

### 4.1.3 Cold Allodynia

The administration of L-29 at all doses did not yield a statistically significant reversal of cold allodynia associated with partial sciatic nerve ligation. However, there was a trend in this direction in that the mean percentage of positive responses decreased in all dose groups during the course of the experiment. A maximum effect was observed at doses of 1 and 10 mgkg⁻¹ between 40-60 minutes post i.p. drug administration with a decline from 100% positive response to 50%. A decline by almost half was similarly observed at a dose of 0.1 mgkg⁻¹ between 20-40 minutes. (FIG. 6).

### 4.2 Receptor Involvement

The effect of selective CB₁ and CB₂ receptor antagonists on L-29 effects was investigated at a dose of 1 mgkg⁻¹ L-29.

### 4.2.1 CB₁ Receptor Antagonist (SR141716a)

Co-administration of this selective antagonist at the CB₁ receptor appeared not to prevent the anti-hyperalgesic and anti-allodynic effects seen at the 1 mgkg⁻¹ dose of L-29. Both mechanical allodynia and thermal hyperalgesia were reversed throughout the entire experiment with sensory thresholds raised significantly above baseline. A significant reduction in % positive response to cold stimulus and hence reversal of cold allodynia occurred at the end of the 80 minute experiment. However, a downward trend in % positive response to cold stimulus may be observed from start of the experiment. (FIG. 10).

### 4.2.2 CB₂ Receptor Antagonist (SR144528)

The effect of co-administration of this selective antagonist at the CB₂ receptor appears more difficult to interpret. It seems to have prevented the anti-allodynic effects of 1 mg/kg L-29 to mechanical stimuli only, these effects being observed from 40 minutes post drug administration. However, there appears to be no reversal of the anti-hyperalgesic effects of i.p. L-29 to thermal stimuli, nor any reversal of cold allodynia (FIG. 11).

### 5. Discussion

This study provides evidence for analgesic actions of palmitoylallylamide (L-29) at doses of 1 and 10 mgkg⁻¹ in a model of peripheral nerve injury related neuropathy, although large group sizes would be required to be certain of the effects demonstrated here. This supports previous work in a rat model of formalin-evoked inflammatory pain in which both 1 and 10 mgkg⁻¹ doses L-29 significantly attenuated pain behaviour, while 0.1 mgkg⁻¹ had no effect¹.

Overall, mechanical allodynia produced by partial sciatic nerve ligation was not as effectively attenuated as thermal hyperalgesia in which sensory thresholds were increased significantly throughout the entire experiment. In addition, no significant reversal of cold allodynia was observed.

In this study, it appears that only the anti-allodynic effect of L-29 to mechanical stimuli is antagonised by the CB₂ receptor antagonist SR144528, and not by the CB₁ receptor antagonist SR141716a as previous work has suggested^{1,5}. In the formalin model of inflammatory pain, the selective CB₁ receptor antagonist, SR141716a (1 mgkg⁻¹) significantly attenuated the anti-nociception produced by L-29 in phase two of the formalin test, while the selective CB₂ receptor antagonist, SR144528 (1 mgkg⁻¹) had no effect, thereby supporting the hypothesis that either fatty acid amide hydrolase inhibitors (FAAHI) induce anti-nociception by a CB₁ receptor mediated mechanism or by interacting with a CB₂-like receptor at which SR144528 is also an antagonist. Similarly, a study investigating the analgesic properties of the synthetic cannabinoid, WIN55, 212-2 in a model of neuropathic pain, found that co-administration of SR141716a but not SR144528 prevented analgesic effects, suggesting that the action of WIN55,212-2 is mediated via the CB₁ receptor⁵.

### REFERENCE LIST

1. Helen Jones. The effect of Palmitoylallylamide, a fatty acid amide hydrolase inhibitor, on formalin-evoked pain in the rat. 2003. ICSM. 2002.
2. Seltzer, Z., Dubner, R. & Shir, Y. A novel behavioral model of neuropathic pain disorders produced in rats by partial sciatic nerve injury. Pain 43, 205-218 (1990).
3. Carlton, S. M., Lekan, H. A., Kim, S. H. & Chung, J. M. Behavioral manifestations of an experimental model for peripheral neuropathy produced by spinal nerve ligation in the primate. Pain 56, 155-166 (1994).
4. Hargreaves, K., Dubner, R., Brown, F., Flores, C. & Joris, J. A new and sensitive method for measuring thermal nociception in cutaneous hyperalgesia. Pain 32, 77-88 (1988).
5. Bridges, D., Ahmad, K. & Rice, A. S. The synthetic cannabinoid WIN55, 212-2 attenuates hyperalgesia and allodynia in a rat model of neuropathic pain. Br. J. Pharmacol. 133, 586-594 (2001).

1-18. (canceled)

19. A method of providing pain relief to a patient comprising administering to said patient an effective amount of a compound of formula I:
RC(O)—NH—(CH₂)ₙ—CH═CH₂
wherein R represents C₁₋₂₀ alkyl, C₂₋₂₀ alkenyl or C₂₋₂₀ alkynyl; and n is an integer from 0 to 3.

20. The method of claim 19 further comprising administering one or more analgesics to the patient.

21. The method of claim 19 wherein, in the compound of formula I, R represent C₁₀₋₂₀ alkyl, C₁₀₋₂₀ alkenyl, or C₁₀₋₂₀ alkynyl.

22. The method of claim 19 wherein, in the compound of formula I, R represents C₁₀₋₂₀ n-alkyl, C₁₀₋₂₀ mono-alkenyl or C₁₀₋₂₀ mono-alkynyl.

23. The method of claim 19 wherein, in the compound of formula I, R represents C₁₀₋₂₀ n-alkyl, C₁₀₋₂₀ mono-n-alkenyl or C₁₀₋₂₀ mono-n-alkynyl.

24. The method of claim 19 wherein, in the compound of formula I, R represents C₁₁₋₁₉ n-alkyl, or C₁₁₋₁₉ mono-n-alkenyl.

25. The method of claim 19 wherein, in the compound of formula I, R represents C₁₁₋₁₈ n-alkyl, or C₁₁₋₁₈ mono-n-alkenyl.

26. The method of claim 19 wherein, in the compound of formula I, the alkenyl or alkynyl groups have no more than 3 C—C double or triple bonds, respectively.

27. The method of claim 19 wherein, in the compound of formula I, n represents 0 or 1.

28. The method of claim 19 wherein, in the compound of formula I, n represents 1.

29. The method of claim 19 wherein the compound is N-(2-propenyl)hexadecanamide, N-(2-propenyl)cis-9-octadecenamide, N-(2-propenyl)cis-9-hexadecenamide, N-(2-propenyl)tetradecanamide, N-(2-propenyl)cis-9-tetradecenamide, N-(2-propenyl)octadecanamide, N-(2-propenyl)trans-9-octadecenamide, N-(2-propenyl) dodecanamide, or N-(2-propenyl)cis-5-dodecenamide.

30. The method of claim 29 wherein the compound is N-(2-propenyl)hexadecanamide.

31. The method of claim 31, wherein the compound is not N-(2-propenyl)-5,8,11,14-eicosatetraenamide.

32. The method of claim 20, wherein the analgesic is an opioid, a non-steroidal anti-inflammatory drug, a local anaesthetic, a NMDA receptor antagonist, a cannabinoid, an antidepressant, and/or an anticonvulsant.

33. A pharmaceutical composition comprising a compound as defined in claim 19 and one or more analgesics and a pharmaceutically acceptable excipient.

34. The pharmaceutical composition of claim 33, wherein the compound is N-(2-propenyl)hexadecanamide, N-(2-propenyl)cis-9-octadecenamide, N-(2-propenyl)cis-9-hexadecenamide, N-(2-propenyl)tetradecanamide, N-(2-propenyl)cis-9-tetradecenamide, N-(2-propenyl)octadecanamide, N-(2-propenyl)trans-9-octadecenamide, N-(2-propenyl)dodecanamide, or N-(2-propenyl)cis-5-dodecenamide.

35. The pharmaceutical composition of claim 33, wherein the analgesic is an opioid, a non-steroidal anti-inflammatory drug, a local anaesthetic, a NMDA receptor antagonist, a cannabinoid, an antidepressant, and/or an anticonvulsant.

36. A kit of parts comprising:
(a) a compound as defined in claim 19; and,
(b) one or more analgesics; and,
(c) a pharmaceutically acceptable excipient.

37. The kit of claim 36, wherein the compound is N-(2-propenyl)hexadecanamide, N-(2-propenyl)cis-9-octadecenamide, N-(2-propenyl)cis-9-hexadecenamide, N-(2-propenyl)tetradecanamide, N-(2-propenyl)cis-9-tetradecenamide, N-(2-propenyl)octadecanamide, N-(2-propenyl)trans-9-octadecenamide, N-(2-propenyl)dodecanamide, or N-(2-propenyl)cis-5-dodecenamide.

38. The kit of claim 37, wherein the analgesic is an opioid, a non-steroidal anti-inflammatory drug, a local anaesthetic, a NMDA receptor antagonist, a cannabinoid, an antidepressant, and/or an anticonvulsant.
